(19) United States
(12) Patent Application Publication
(10) Pub. No.: US 2015/0330926 A1
(43) Pub. Date: Nov. 19, 2015

(54) HAND-HELD TEST METER CONSTANT CURRENT DRIVER WITH INTEGRATED TEST STRIP SAMPLE DETECTION

(71) Applicant: Cilag GmbH International, Zug (CH)

(72) Inventors: David Elder, Inverness (GB); Tim Lloyd, Avoch (GB)

(73) Assignee: Cilag GmbH International, Zug (CH)

(21) Appl. No.: 14/138,549

(22) Filed: Dec. 23, 2013

(51) Int. Cl. G01N 27/02; G01N 33/49

(52) U.S. Cl. CPC G01N 27/021; G01N 33/49

(57) ABSTRACT

A hand-held test meter for the determination of an analyte in a bodily fluid sample using an analytical test strip includes a microprocessor block (MB), a strip port connector (SPC), a voltage driver block (VDB) operatively connected to the MB and the SPC, a current measurement block (CMB) operatively connected to the SPC and the MB, and a memory block operatively coupled to the MB and storing integrated test strip detection and constant current driver instructions. Moreover, the memory block, MB, VDB and CMB are configured such that the integrated test strip detection and constant current driver instructions, when executed by the MB, algorithmically detects sample application to a test strip inserted in the SPC and algorithmically drives a constant current through the inserted strip by varying a voltage applied to the SPC by the VDB based on a signal from the CMB.

### BACKGROUND

Analyte detection in physiological fluids, e.g. blood or blood derived products, is of ever increasing importance to today's society. Analyte detection assays find use in a variety of applications, including clinical laboratory testing, home testing, etc., where the results of such testing play a prominent role in diagnosis and management in a variety of disease conditions. Analytes of interest include glucose for diabetes management, cholesterol, and the like. In response to this growing importance of analyte detection, a variety of analyte detection protocols and devices for both clinical and home use have been developed.

One type of method that is employed for analyte detection is an electrochemical method. In such methods, an aqueous liquid sample is placed into a sample-receiving chamber in an electrochemical cell that includes two electrodes, e.g., a counter and working electrode. The analyte is allowed to react with a redox reagent to form an oxidizable (or reducible) substance in an amount corresponding to the analyte concentration. The quantity of the oxidizable (or reducible) substance present is then estimated electrochemically and related to the amount of analyte present in the initial sample.

Such systems are susceptible to various modes of inefficiency or error.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements).

FIG. 1A illustrates an exemplary glucose measurement system.

FIG. 1B illustrates the various components disposed in the meter of FIG. 1A.

FIG. 1C illustrates a perspective view of an assembled test strip suitable for use in the system and methods disclosed herein;

FIG. 1D illustrates an exploded perspective view of an unassembled test strip suitable for use in the system and methods disclosed herein;

FIG. 1E illustrates an expanded perspective view of a proximal portion of the test strip suitable for use in the system and methods disclosed herein;

FIG. 2 is a bottom plan view of one embodiment of a test strip disclosed herein;

FIG. 3 is a side plan view of the test strip of FIG. 2;

FIG. 4A is a top plan view of the test strip of FIG. 3;

FIG. 4B is a partial side view of a proximal portion of the test strip of FIG. 4A;

FIG. 5 is a simplified schematic showing a test meter electrically interfacing with portions of a test strip disclosed herein;

FIG. 6A shows an example of a tri-pulse potential waveform applied by the test meter of FIG. 5 to the working and counter electrodes for prescribed time intervals;

FIG. 6B shows a current transient CT generated by a physiological sample;

FIG. 7 is a simplified block diagram of a hand-held test meter according to an embodiment of the present invention;

FIG. 8 is a simplified flow chart (with annotations) for a sequence of steps for a constant current driver with integrated test strip sample detection as can be employed in embodiments of the present invention;

FIG. 9 is a chart depicting the voltage applied to an SPC by an algorithm as can be employed in embodiments of the present invention (labeled Drive V S/W) in comparison to the voltage applied to an SPC using conventional hardware-only driven techniques (marked Drive V H/W); and

FIG. 10 is a flow diagram depicting stages in a method for operating a hand-held test meter according to an embodiment of the present invention that can, for example, utilize the flow chart of FIG. 8.

### MODES FOR CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, as used herein, the terms “patient,” “host,” “user,” and “subject” refer to any human or animal subject and are not intended to limit the systems or methods to human use, although use of the subject invention in a human patient represents a preferred embodiment. Also used herein, the phrase “electrical signal” or “signal” is intended to include direct current signal, alternating signal or any signal within the electromagnetic spectrum. The terms “processor”; “microprocessor”; or “microcontroller” are intended to have the same meaning and may be used interchangeably. As used herein, the term “annunciated” and variations on its root term indicate that an announcement may be provided via text, audio, visual or a combination of all modes or mediums of communication to a user.

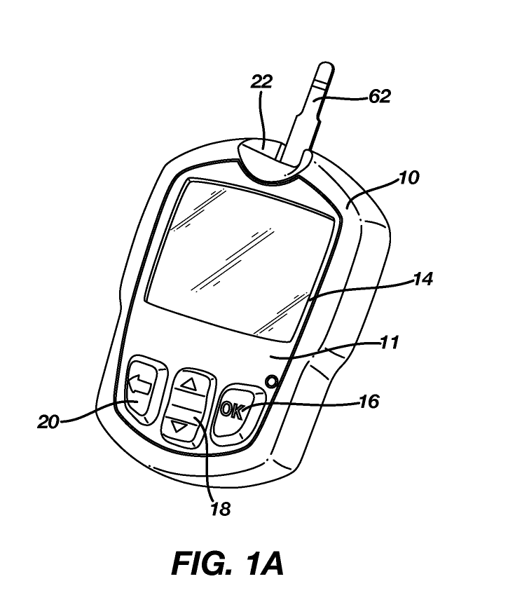

FIG. 1A illustrates a diabetes management system that includes a meter 10 and a biosensor in the form of a glucose test strip 62. Note that the meter (or meter unit) may be referred to as an analyte measurement and management unit, a glucose meter, a meter, and an analyte measurement device. In an embodiment, the meter unit may be combined with an insulin delivery device, an additional analyte testing device, and a drug delivery device. The meter unit may be connected to a remote computer or remote server via a cable or a suitable wireless technology such as, for example, GSM, CDMA, Bluetooth, WiFi and the like.

Referring back to FIG. 1A, glucose meter or meter unit 10 may include a housing 11, user interface buttons (16, 18, and 20), a display 14, and a strip port opening 22 to receive a biosensor or strip 62. User interface buttons (16, 18, and 20) may be configured to allow the entry of data, navigation of menus, and execution of commands. User interface button 18 may be in the form of a two-way toggle switch. Alternatively, the buttons may be replaced with a touch-screen interface for display 14. Data may include values representative of analyte concentration, or information related to the everyday lifestyle of an individual. Such information may include food intake, medication use, occurrence of health check-ups, and general health condition and exercise levels of an individual.

FIG. 1B illustrates (in simplified schematic form) the electronic components disposed on a top surface of circuit board 34, which is disposed in housing 11 (FIG. 1A). On the top surface, the electronic components include a strip port connector 22, an operational amplifier circuit 35, a microcontroller 38, a display connector 14a, a non-volatile memory 40, a clock 42, and a first wireless module 46. On the bottom surface, the electronic components may include a battery connector (not shown) and a data port 13. Microcontroller 38 may be connected to strip port connector 22, operational amplifier circuit 35, first wireless module 46, display 14, non-volatile memory 40, clock 42, battery, data port 13, and user interface buttons (16, 18, and 20).

Operational amplifier circuit 35 may include two or more operational amplifiers configured to provide a portion of the potentiostat function and the current measurement function. The potentiostat function may refer to the application of a test voltage between at least two electrodes of a test strip. The current function may refer to the measurement of a test current resulting from the applied test voltage. The current measurement may be performed with a current-to-voltage converter. Microcontroller 38 may be in the form of a mixed signal microprocessor (MSP) such as, for example, the Texas Instrument MSP430. The TI MSP430 may be configured to also perform a portion of the potentiostat function and the current measurement function. In addition, the MSP430 may also include volatile and non-volatile memory. In another embodiment, many of the electronic components may be integrated with the microcontroller in the form of an application specific integrated circuit (ASIC).

Strip port connector 22 may be configured to form an electrical connection to the test strip. Display connector 14a may be configured for attachment to display 14. Display 14 may be in the form of a liquid crystal display for reporting measured glucose levels, and for facilitating entry of lifestyle related information. Display 14 may also include a backlight. Data port 13 may accept a suitable connector attached to a connecting lead, thereby allowing glucose meter 10 to be linked to an external device such as a personal computer. Data port 13 may be any port that allows for transmission of data such as, for example, a serial, USB, or a parallel port. Alternatively, wireless module 46 may also be used in place of the data port and connector to transfer data to another device. Clock 42 may be configured to keep current time related to the geographic region in which the user is located and also for measuring time. The meter unit may be configured to be electrically connected to a power supply such as, for example, a battery.

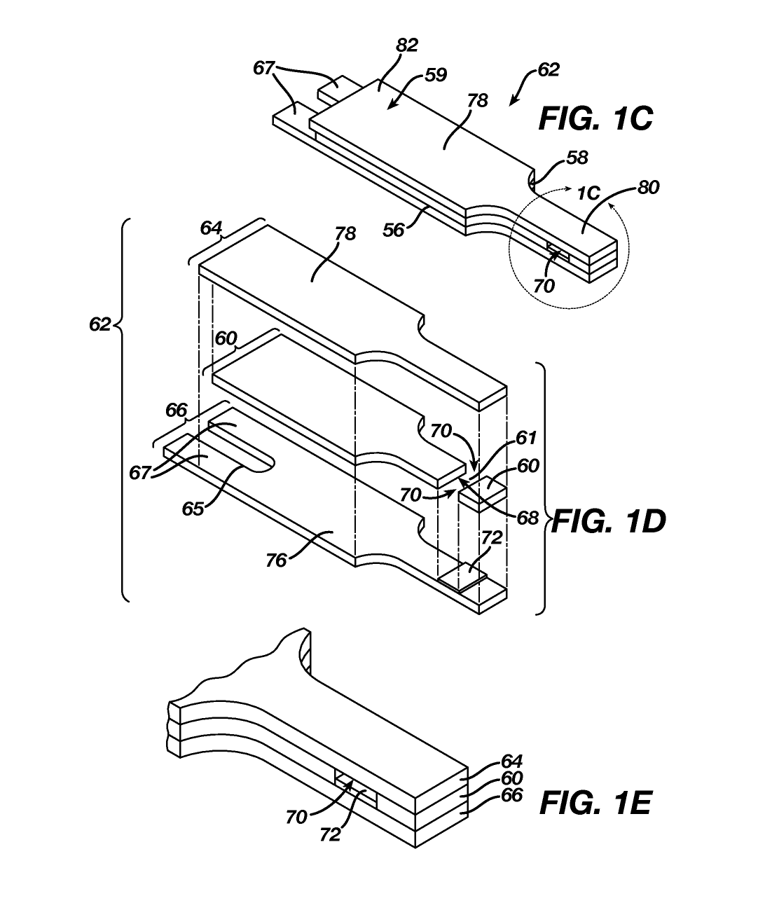

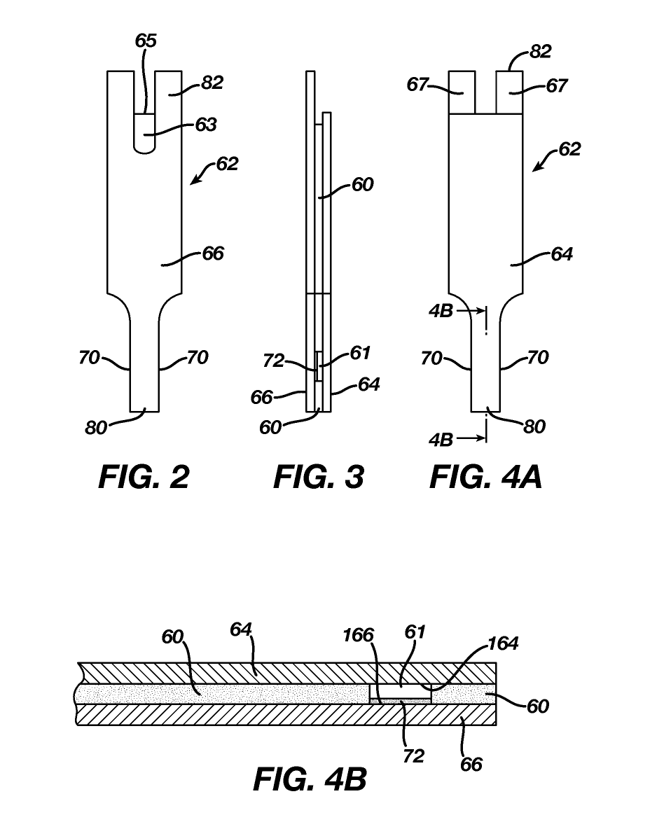

FIGS. 1C-1E, 2, 3, and 4B show various views of an exemplary test strip 62 suitable for use with the methods and systems described herein. In an exemplary embodiment, a test strip 62 is provided which includes an elongate body extending from a distal end 80 to a proximal end 82, and having lateral edges 56, 58, as illustrated in FIG. 1C. As shown in FIG. 1D, the test strip 62 also includes a first electrode layer 66, a second electrode layer 64, and a spacer 60 sandwiched in between the two electrode layers 64 and 66. The first electrode layer 66 may include a first electrode 66, a first connection track 76, and a first contact pad 67, where the first connection track 76 electrically connects the first electrode 66 to the first contact pad 67, as shown in FIGS. 1D and 4B. Note that the first electrode 66 is a portion of the first electrode layer 66 that is immediately underneath the reagent layer 72, as indicated by FIGS. 1D and 4B. Similarly, the second electrode layer 64 may include a second electrode 64, a second connection track 78, and a second contact pad 63, where the second connection track 78 electrically connects the second electrode 64 with the second contact pad 63, as shown in FIGS. 1D, 2, and 4B. Note that the second electrode 64 is a portion of the second electrode layer 64 that is above the reagent layer 72, as indicated by FIG. 4B.

As shown in FIGS. 1D and 4B, the sample-receiving chamber 61 is defined by the first electrode 66, the second electrode 64, and the spacer 60 near the distal end 80 of the test strip 62. The first electrode 66 and the second electrode 64 may define the bottom and the top of sample-receiving chamber 61, respectively, as illustrated in FIG. 4B. As illustrated in FIG. 4B A, a cutout area 68 of the spacer 60 may define the sidewalls of the sample-receiving chamber 61. In one aspect, the sample-receiving chamber 61 may include ports 70 that provide a sample inlet or a vent, as shown in FIGS. 1C to 1E. For example, one of the ports may allow a fluid sample to ingress and the other port may allow air to egress.

In an exemplary embodiment, the sample-receiving chamber 61 (also known as a “test cell” or “test chamber”) may have a small volume. For example, the chamber 61 may have a volume in the range of from about 0.1 microliters to about 5 microliters, about 0.2 microliters to about 3 microliters, or, preferably, about 0.3 microliters to about 1 microliter. To provide the small sample volume, the cutout 68 may have an area ranging from about 0.01 cm² to about 0.2 cm², about 0.02 cm² to about 0.15 cm², or, preferably, about 0.03 cm² to about 0.08 cm². In addition, first electrode 66 and second electrode 64 may be spaced apart in the range of about 1 micron to about 500 microns, preferably between about 10 microns and about 400 microns, and more preferably between about 40 microns and about 200 microns. The relatively close spacing of the electrodes may also allow redox cycling to occur, where oxidized mediator generated at first electrode 66, may diffuse to second electrode 64 to become reduced, and subsequently diffuse back to first electrode 66 to become oxidized again.

In one embodiment, the first electrode layer 66 and the second electrode layer 64 may be a conductive material formed from materials such as gold, palladium, carbon, silver, platinum, tin oxide, iridium, indium, or combinations thereof (e.g., indium doped tin oxide). In addition, the electrodes may be formed by disposing a conductive material onto an insulating sheet (not shown) by a sputtering, electroless plating, or a screen-printing process. In one exemplary embodiment, the first electrode layer 66 and the second electrode layer 64 may be made from sputtered palladium and sputtered gold, respectively. Suitable materials that may be employed as spacer 60 include a variety of insulating materials, such as, for example, plastics (e.g., PET, PETG, polyimide, polycarbonate, polystyrene), silicon, ceramic, glass, adhesives, and combinations thereof. In one embodiment, the spacer 60 may be in the form of a double-sided adhesive coated on opposing sides of a polyester sheet where the adhesive may be pressure sensitive or heat activated. Various other materials for the first electrode layer 66, the second electrode layer 64, or the spacer 60 are within the spirit and scope of the present disclosure.

Either the first electrode 66 or the second electrode 64 may perform the function of a working electrode depending on the magnitude or polarity of the applied test voltage. The working electrode may measure a limiting test current that is proportional to the reduced mediator concentration. For example, if the current limiting species is a reduced mediator (e.g., ferrocyanide), then it may be oxidized at the first electrode 66 as long as the test voltage is sufficiently greater than the redox mediator potential with respect to the second electrode 64. In such a situation, the first electrode 66 performs the function of the working electrode and the second electrode 64 performs the function of a counter/reference electrode. Applicants note that one may refer to a counter/reference electrode simply as a reference electrode or a counter electrode. A limiting oxidation occurs when all reduced mediator has been depleted at the working electrode surface such that the measured oxidation current is proportional to the flux of reduced mediator diffusing from the bulk solution towards the working electrode surface. The term “bulk solution” refers to a portion of the solution sufficiently far away from the working electrode where the reduced mediator is not located within a depletion zone. It should be noted that unless otherwise stated for test strip 62, all potentials applied by test meter 10 will hereinafter be stated with respect to second electrode 64.

Similarly, if the test voltage is sufficiently less than the redox mediator potential, then the reduced mediator may be oxidized at the second electrode 64 as a limiting current. In such a situation, the second electrode 64 performs the function of the working electrode and the first electrode 66 performs the function of the counter/reference electrode.

Initially, an analysis may include introducing a quantity of a fluid sample (e.g., physiological fluid sample or calibration fluid) into a sample-receiving chamber 61 via a port 70 (FIG. 1C). In one aspect, the port 70 or the sample-receiving chamber 61 may be configured such that capillary action causes the fluid sample to fill the sample-receiving chamber 61. The first electrode 66 or second electrode 64 may be coated with a hydrophilic reagent to promote the capillary action of the sample-receiving chamber 61. For example, thiol derivatized reagents having a hydrophilic moiety such as 2-mercaptoethane sulfonic acid may be coated onto the first electrode or the second electrode to provide for such action.

In strip 62 above, reagent layer 72 can include glucose dehydrogenase (GDH) based on the PQQ co-factor and ferricyanide. In another embodiment, the enzyme GDH based on the PQQ co-factor may be replaced with the enzyme GDH based on the FAD co-factor. When physiological fluid containing glucose (e.g., blood or control solution) is dosed into a sample reaction chamber 61, glucose is oxidized by GDH₍ₒₓ₎ and in the process converts GDH₍ₒₓ₎ to GDH_{(red)}, as shown in the chemical reaction or transformation T.1 below. Note that GDH₍ₒₓ₎ refers to the oxidized state of GDH, and GDH_{(red)} refers to the reduced state of GDH.

D-Glucose+GDH₍ₒₓ₎→Gluconic acid+GDH_{(red)} T.1

Next, GDH_{(red)} is regenerated back to its active oxidized state by ferricyanide (i.e. oxidized mediator or Fe(CN)₆³⁻) as shown in chemical reaction T.2 below. In the process of regenerating GDH₍ₒₓ₎, ferrocyanide (i.e. reduced mediator or Fe(CN)₆⁴⁻) is generated from the reaction as shown in T.2:

GDH_{(red)}+2Fe(CN)₆³⁻→GDH₍ₒₓ₎+2Fe(CN)₆⁴⁻ T.2

Ferrocyanide generated by transformation T2 causes an electrical current to flow through the electrodes on the biosensor. The more glucose is in the fluid sample, the more gluconic acid is produced in transformation T1, increasing the electrical current generated by ferrocyanide in transformation T2.

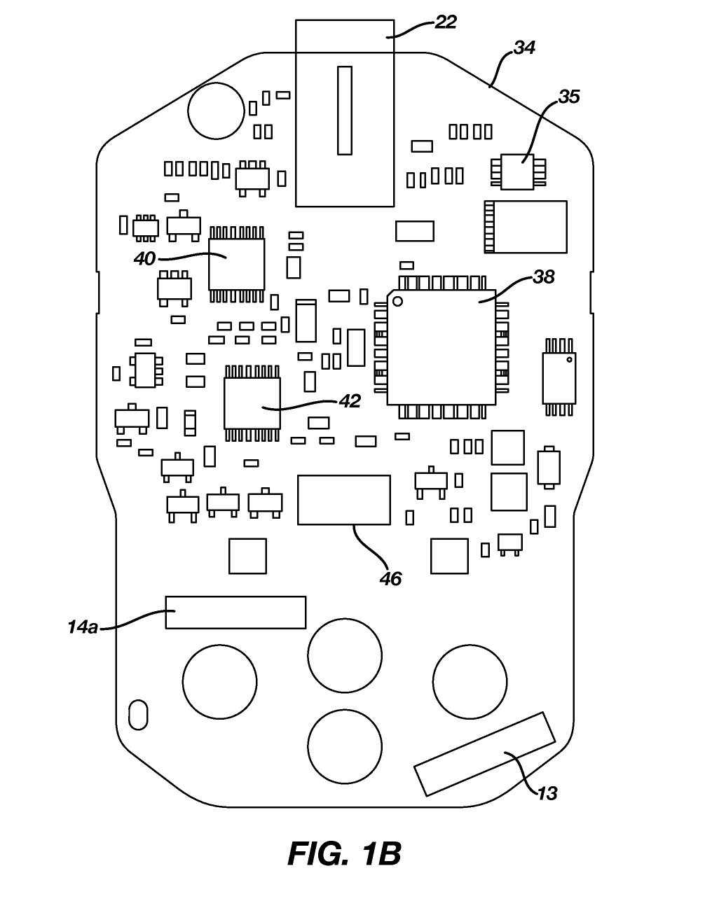

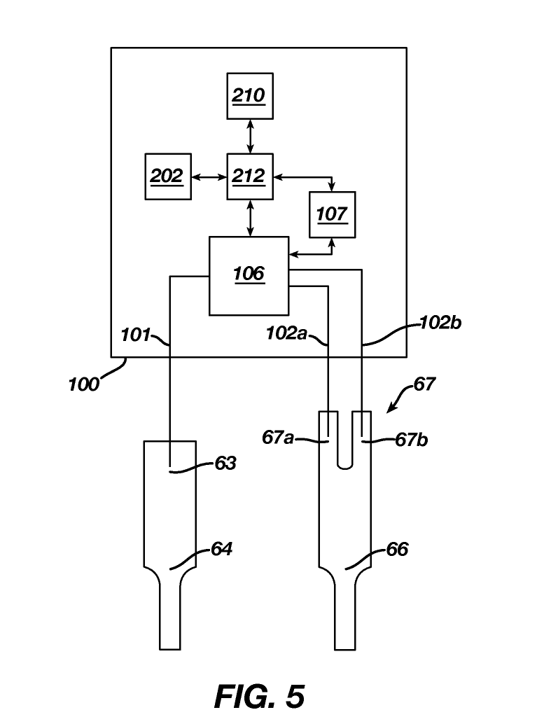

FIG. 5 provides a simplified schematic of test meter 10 in the form of measurement module 100 interfacing with a first contact pad 67a, 67b and a second contact pad 63. The second contact pad 63 may be used to establish an electrical connection to the test meter through a U-shaped notch 65, as illustrated in FIG. 2. In one embodiment, the measurement module 100 may include a first electrode connectors (102a, 102b) and a second electrode connector 101 with a test voltage unit 106, a current measurement unit 107, a processor 212, a memory unit 210, and a visual display 202, as shown in FIG. 5. The first contact pad 67 may include two prongs denoted as 67a and 67b. In one exemplary embodiment, the first electrode connectors 102a and 102b separately connect to prongs 67a and 67b, respectively. The second electrode connector 101 may connect to second contact pad 63. The measurement module 100 may measure the resistance or electrical continuity between the prongs 67a and 67b to determine whether the test strip 62 is electrically connected to the test meter 10.

Meter 10 (FIGS. 1A, 1B) may include electronic circuitry that can be used to apply a plurality of voltages to the test strip 62 and to measure a current transient output resulting from an electrochemical reaction in a test chamber of the test strip 62. Meter 10 also may include a set of instructions programmed into the microprocessor to determine an analyte concentration in a fluid sample as disclosed herein.

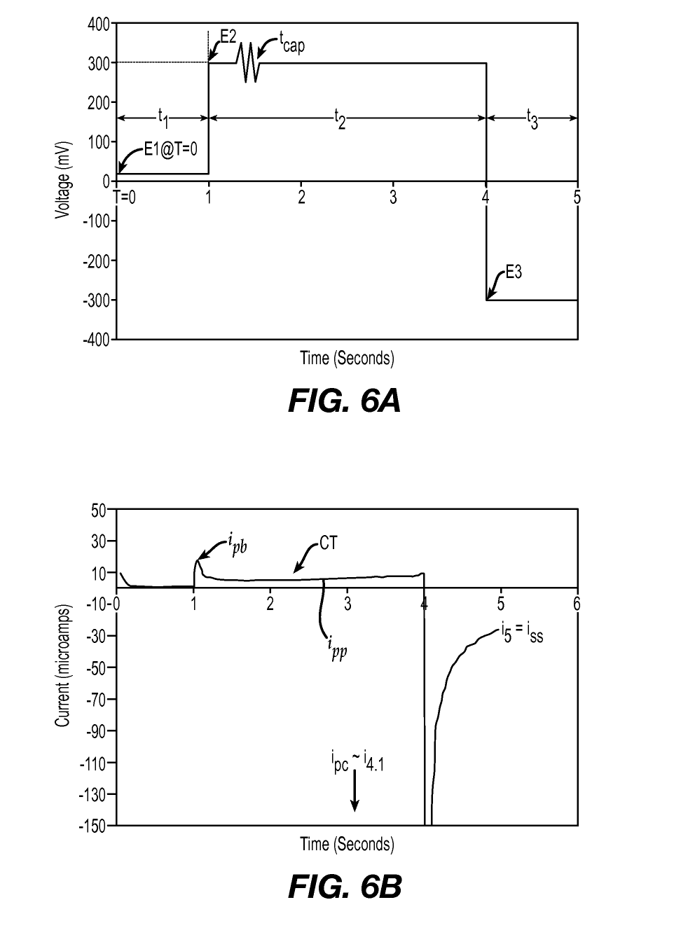

In use, the user inserts the test strip into a strip port connector of the test meter 10 to connect at least two electrodes of the test strip to a strip measurement circuit. This turns on the meter 10 and meter 10 (via module 100) may apply a test voltage or a current between the first contact pad 67 and the second contact pad 63 (FIG. 5). Once the measurement module 100 recognizes that the strip 62 has been inserted, the measurement module 100 initiates a fluid detection mode. The fluid detection mode causes measurement module 100 to apply a constant current of about 1 microampere between the first electrode 66 and the second electrode 64. Because the test strip 62 is initially dry, the test meter 10 measures a relatively large voltage. When the fluid sample is deposited onto the test chamber, the sample bridges the gap between the first electrode 66 and the second electrode 64 and the measurement module 100 will measure a decrease in measured voltage that is below a predetermined threshold. This causes test meter 10 to automatically initiate the glucose test by application of a first electrical potential E1 (FIG. 6A).

In FIG. 6A (which has its time axis in alignment with the time axis of FIG. 6B), the analyte in the sample is transformed from one form (e.g., glucose) into a different form (e.g., gluconic acid) due to an electrochemical reaction in the test chamber that starts with initiation of the test sequence at T=0 by a test sequence timer, which timer is set by a detection of strip fill and setting the potential at E1 for a first duration of t₁. The system proceeds through the test sequence by switching the first electrical potential from E1 to a second electrical potential E2 different than the first electrical potential E1 (FIG. 6A) for a second duration t₂, then the system further changes the second potential E2 to a third potential E3 different from the second electrical potential E2 (FIG. 6A) for a third duration t₃. The third electrical potential E3 may be different in the magnitude of the electromotive force, in polarity, or combinations of both with respect to the second electrical potential E2. In the preferred embodiments, E3 may be of the same magnitude as E2 but opposite in polarity.

Further, as illustrated in FIG. 6A, the second electrical potential E2 may include a direct (DC) test voltage component and a superimposed alternating (AC), or alternatively oscillating, test voltage component. The superimposed alternating or oscillating test voltage component may be applied for a time interval indicated by t_{cap}. This superimposed alternating voltage is utilized to determine if the strip has sufficient volume of the fluid sample in which to conduct a test. Details of this technique to determine sufficient volume for electrochemical testing are shown and described in U.S. Pat. Nos. 7,195,704; 6,872,298, 6,856,125, 6,797,150, which documents are incorporated by reference as if fully set forth herein.

The plurality of test current values measured during any of the time intervals may be performed at a sampling frequency ranging from about 1 measurement per microsecond to about one measurement per 100 milliseconds and preferably at about every 10 to 50 milliseconds. While an embodiment using three test electrical potentials in a serial manner is described, the glucose test may include different numbers of open-circuit and test voltages. For example, as an alternative embodiment, the glucose test could include an open-circuit for a first time interval, a second test voltage for a second time interval, and a third electrical potential for a third time interval. It should be noted that the reference to “first,” “second,” and “third” are chosen for convenience and do not necessarily reflect the order in which the test voltages are applied. For instance, an embodiment may have a potential waveform where the third electrical potential may be applied before the application of the first and second test voltages.

In this exemplary system, the process for the system may apply a first electrical potential E1 (e.g., approximately 20 mV in FIG. 6A) between first electrode 66 and second electrode 64 for a first time interval t₁ (e.g., 1 second in FIG. 6A). The first time interval t₁ may range from about 0.1 seconds to about 3 seconds and preferably range from about 0.2 seconds to about 2 seconds, and most preferably range from about 0.3 seconds to about 1.1 seconds.

The first time interval t₁ may be sufficiently long so that the sample-receiving or test chamber 61 may fully fill with sample and also so that the reagent layer 72 may at least partially dissolve or solvate. In one aspect, the first electrical potential E1 may be a value relatively close to the redox potential of the mediator so that a relatively small amount of a reduction or oxidation current is measured. FIG. 6B shows that a relatively small amount of current is observed during the first time interval t₁ compared to the second and third time intervals t₂ and t₃ for FIG. 6A. For example, when using ferricyanide or ferrocyanide as the mediator, the first electrical potential E1 in FIG. 6A may range from about 1 mV to about 100 mV, preferably range from about 5 mV to about 50 mV, and most preferably range from about 10 mV to about 30 mV. Although the applied voltages are given as positive in polarity in the preferred embodiments, the same voltages in the negative domain could also be utilized to accomplish the intended purpose of the present embodiments.

Referring back to FIG. 6A, after applying the first electrical potential E1, the test meter 10 applies a second electrical potential E2 between first electrode 66 and second electrode 64 (e.g., approximately 300 mVolts in FIG. 6A), for a second time interval t₂ (e.g., about 3 seconds in FIG. 6A). The second electrical potential E2 may be a value different than the first electrical potential E1 and may be sufficiently negative of the mediator redox potential so that a limiting oxidation current is measured at the second electrode 64. For example, when using ferricyanide or ferrocyanide as the mediator, the second electrical potential E2 may range from about zero mV to about 600 mV, preferably range from about 100 mV to about 600 mV, and more preferably is about 300 mV.

The second time interval t₂ should be sufficiently long so that the rate of generation of reduced mediator (e.g., ferrocyanide) may be monitored based on the magnitude of a limiting oxidation current. Reduced mediator is generated by enzymatic reactions with the reagent layer 72. During the second time interval t₂, a limiting amount of reduced mediator is oxidized at second electrode 64 and a non-limiting amount of oxidized mediator is reduced at first electrode 66 to form a concentration gradient between first electrode 66 and second electrode 64.

In an exemplary embodiment, the second time interval t₂ should also be sufficiently long so that a sufficient amount of ferricyanide may be diffused to the second electrode 64 or diffused from the reagent on the first electrode. A sufficient amount of ferricyanide is required at the second electrode 64 so that a limiting current may be measured for oxidizing ferrocyanide at the first electrode 66 during the third electrical potential E3. The second time interval t₂ may be less than about 60 seconds, and preferably may range from about 1.1 seconds to about 10 seconds, and more preferably range from about 2 seconds to about 5 seconds. Likewise, the time interval indicated as t_{cap} in FIG. 6A may also last over a range of times, but in one exemplary embodiment, it has a duration of about 20 milliseconds. In one exemplary embodiment, the superimposed alternating test voltage component is applied after about 0.3 seconds to about 0.4 seconds after the application of the second electrical potential E2, and induces a sine wave having a frequency of about 109 Hz with an amplitude of about +/−50 mV.

FIG. 6B shows a relatively small peak i_{pb} after the beginning of the second time interval t₂ followed by a gradual increase of an absolute value of an oxidation current during the second time interval t₂. The small peak i_{pb} occurs due oxidation of endogenous or exogenous reducing agents (e.g., uric acid) after a transition from first electrical potential E1 to second electrical potential E2. Thereafter, there is a gradual absolute decrease in oxidation current after the small peak i_{pb}. This peak is caused by the generation of ferrocyanide by reagent layer 72, which then diffuses to second electrode 64. During the second time interval t2, a current iₚₚ can be measured from the current transient CT in the oxidation current.

After application of the second electrical potential E2, the test meter 10 applies a third electrical potential E3 between the first electrode 66 and the second electrode 64 (e.g., about −300 mVolts in FIG. 6A) for a third time interval t₃ (e.g., 1 second in FIG. 6A). The third electrical potential E3 may be a value sufficiently positive of the mediator redox potential so that a limiting oxidation current is measured at the first electrode 66. For example, when using ferricyanide or ferrocyanide as the mediator, the third electrical potential E3 may range from about zero mV to about −600 mV, preferably range from about −100 mV to about −600 mV, and more preferably is about −300 mV.

The third time interval t₃ may be sufficiently long to monitor the diffusion of reduced mediator (e.g., ferrocyanide) near the first electrode 66 based on the magnitude of the oxidation current. During the third time interval t₃, a limiting amount of reduced mediator is oxidized at first electrode 66 and a non-limiting amount of oxidized mediator is reduced at the second electrode 64. The third time interval t₃ may range from about 0.1 seconds to about 5 seconds and preferably range from about 0.3 seconds to about 3 seconds, and more preferably range from about 0.5 seconds to about 2 seconds.

FIG. 6B shows a relatively large peak i_{pc} at the beginning of the third time interval is followed by a decrease to a steady-state current iₛₛ value. The measured current outputs i_{pb}, i_{pc} iₚₚ and iₛₛ can be used to determine a glucose concentration of the sample from Equation 1:

$$G = {( \frac{{i}_{ss}}{{i}_{pp}} )}^{p} \times ( a \{ \frac{{i}_{pc} + {bi}_{ss} - 2 {i}_{pb}}{{i}_{pc} + {bi}_{ss}} \} {i}_{ss} - Z ) \qquad Equation 1$$

Where G is the glucose concentration;
   iₛₛ is a magnitude of measured signals (in amperage) as a summation from about 4 seconds to about 5 seconds of the current transient
   iₚₚ is a magnitude of measured signals (in amperage) as a summation from about 1 second to about 4 seconds of the current transient;
   i_{pb} is a magnitude of measured signal (in amperage) at about 1 second of the current transient;
   i_{pc} is a magnitude of measured signal (in amperage) at about 4 seconds of the current transient;
   a is about 0.2;
   b is about 0.7
   p is about 0.5; and
   Z is about 4.

Additional details on the biosensor system can be found in U.S. Pat. No. 8,163,162, patented Apr. 24, 2012, which is hereby incorporated by reference in its entirety into this application.

In general, hand-held test meters for the determination of an analyte (such as glucose) in a bodily fluid sample (for example, a whole blood sample) using an analytical test strip (e.g., an electrochemical-based analytical test strip) include a microprocessor block, a strip port connector (SPC), a voltage driver block operatively connected to the microprocessor block and the SPC, a current measurement block operatively connected to the SPC and the micro-processor block, and a memory block operatively coupled to the microprocessor block and storing integrated test strip detection and constant current driver instructions. Moreover, the memory block, microprocessor block, voltage driver block and current measurement block are configured such that the integrated test strip detection and constant current driver instructions, when executed by the microprocessor block, algorithmically detects sample application to a test strip inserted in the SPC and algorithmically drives a constant current through the inserted strip by varying a voltage applied to the SPC by the voltage driver block based on the signal from the current measurement block.

Hand-held test meters according to the present invention are beneficial in that, for example, they drive a constant current across an inserted analytical test strip (e.g., an electrochemical-based analytical test strip) using an algorithmically-based software (i.e., an instruction set that includes an algorithm) in a manner that is integrated with algorithmically-based test strip sample detection. Such integration can include, for example, using a voltage output (or a voltage derived therefrom) from an algorithm of the constant current driver instructions as an input to an algorithm of the test strip detection instructions. The hand held test meters are beneficially simple and relatively inexpensive since they do not employ a hardware-based constant current electronic circuit.

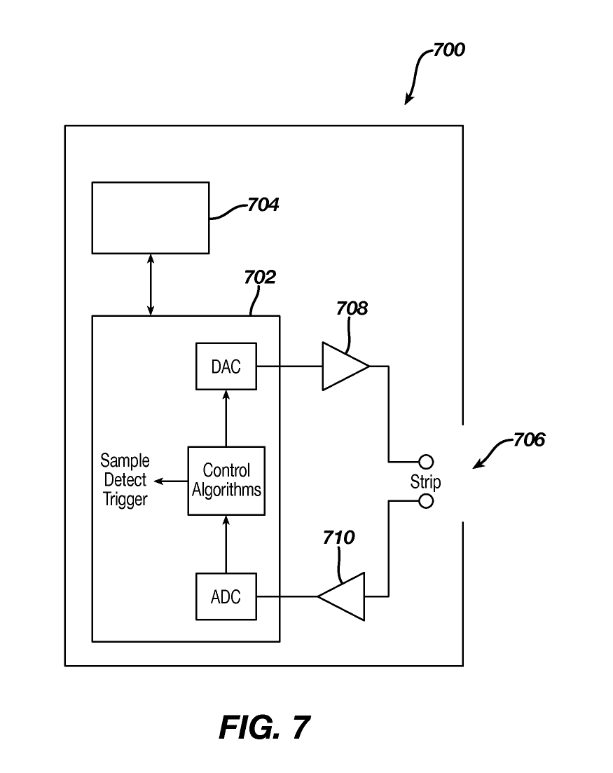

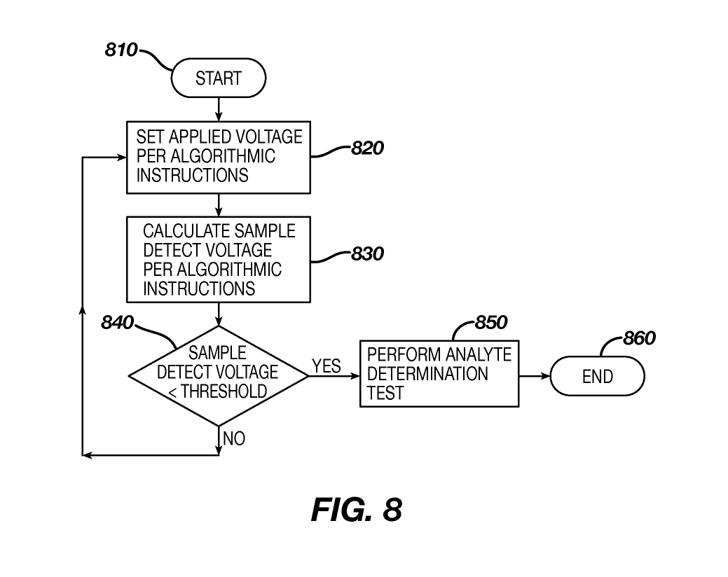

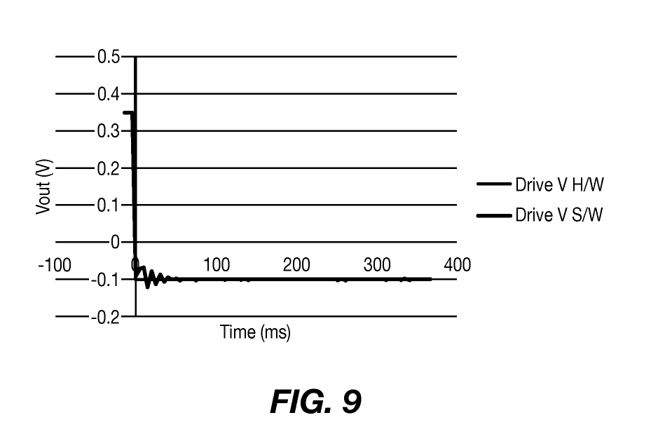

FIG. 7 is a simplified block diagram of a hand-held test meter 700 for the determination of an analyte in a bodily fluid sample according to an embodiment of the present invention. FIG. 8 is a simplified flow chart for a sequence of steps for a constant current driver with integrated test strip sample detection as can be employed in embodiments of the present invention. FIG. 9 is a chart depicting the voltage applied to an SPC by an algorithm as can be employed in embodiments of the present invention (labeled Drive V S/W) in comparison to the voltage applied to an SPC using conventional hardware-only driven techniques (marked Drive V H/W).

Referring to FIGS. 7, 8 and 9, hand-held test meter 700 includes a microprocessor block 702, a memory block 704, a strip port connector 706, a voltage driver block 708 and a current measurement block 710, and other electronic components (not shown) for applying an electrical bias (e.g., an alternating current (AC) and/or direct current (DC) bias) to an electrochemical-based analytical test strip, and also for measuring an electrochemical response (e.g., plurality of test current values, phase, and/or magnitude) and determining an analyte or characteristic based on the electrochemical response.

To simplify the current descriptions, the FIG. 7 does not depict all the electronic circuitry and mechanical blocks of hand-held test meter 700. However, once apprised of the present disclosure, one skilled in the art will recognize that hand-held test meter 700 also includes further blocks and circuits required or desirable for the determination of an analyte (such as glucose) in a bodily fluid sample (for example, a whole blood sample) using, for example, an electrochemical-based analytical test strip (not shown in FIG. 7 but located where the annotation “strip” is located in FIG. 7). Moreover, one skilled in the art will recognize that various separate blocks depicted in FIG. 7 can be integrated in any suitable manner.

Once one skilled in the art is apprised of the present disclosure, he or she will recognize that an example of a hand-held test meter that can be readily modified as a hand-held test meter according to the present invention is the commercially available OneTouch® Ultra® 2 glucose meter from LifeScan Inc. (Milpitas, Calif.). Additional examples of hand-held test meters that can also be modified are found in U.S. Patent Application Publications No's. 2007/0084734 (published on Apr. 19, 2007) and 2007/0087397 (published on Apr. 19, 2007) and in International Publication Number WO2010/049669 (published on May 6, 2010), and Great Britain Patent Application No. 1303616.5, filed on Feb. 28, 2013, each of which is hereby incorporated herein in full by reference.

Microprocessor block 702 can be any suitable microprocessor block known to one skilled in the art including, but not limited to, a micro-controller. Suitable micro-controllers include, but are not limited to, micro-controllers available commercially from Texas Instruments (Dallas, Tex., USA) under the MSP430 series of part numbers; from ST MicroElectronics (Geneva, Switzerland) under the STM32F and STM32L series of part numbers; and Atmel Corporation (San Jose, Calif., USA) under the SAM4L series of part numbers). Microprocessor 702 is shown as including integrated analog-to-digital (ADC) and digital-to-analog (DAC) electrical circuits as well as circuitry configured to execute instructions including algorithmic instructions.

Voltage driver block 708 can be any suitable voltage driver block including, for example, an operational-amplifier voltage driver block. A non-limiting example of a suitable operational-amplifier that can be included in, or serve as, a voltage driver block is the operational amplifier available as part number OPA348 from Texas Instruments, Dallas, Tex., USA.

Current measurement block 710 can be any suitable current measurement block, including a current measurement block based on an operational amplifier. A non-limiting example of a suitable operational-amplifier that can be included in, or serve as, a current measurement block is the operational amplifier available as part number OPA330 from Texas Instruments, Dallas, Tex., USA.

In FIG. 7, both voltage driver block 708 and current measurement block 710 are depicted using a triangular shape. Such a shape typically represents an amplifier. However, one skilled in the art, once apprised of the present disclosure, will recognize that such amplifiers may be combined with various passive devices to operate either as a voltage driver block or a current measurement block using techniques known to those of skill in the art.

Memory block 704 is coupled to the microprocessor block 702 and stores integrated test strip sample detection and constant current driver instructions as described, for example with respect to FIG. 8 and algorithms 1 and 2 below. Memory block 704, microprocessor block 702, voltage driver block 708 and current measurement block 710 are configured such that the integrated test strip sample detection and constant current driver instructions, when executed by the microprocessor block, algorithmically detects sample application to a test strip inserted in the SPC based and algorithmically drives a constant current through the inserted strip by varying a voltage applied to the SPC by the voltage driver block based on a signal from the current measurement block.

The constant current driver instruction can, for example, be based on a feedback loop such as a PID algorithm feedback loop. A non-limiting example of such a PID algorithm employed in the instructions is as follows:

Vₒᵤₜ=(Iₑᵣᵣ*Gₚ)+(Iᵢₙₜ*Gᵢ)(I_{diff}*G_{d}) (Algorithm 1)

where:

Iₑᵣᵣ=difference between a measured current and a predetermined target current (e.g., 300 nA), Iₑᵣᵣ equals 0 when the measured current equals the target current;

Gₚ=proportional gain constant, e.g., 800

Iᵢₙₜ=the sum of previous Ierr values, however if current is greater than a predetermined over-limit current, for example, >432 nA, then Iint=0;
Gᵢ=integral gain constant, e.g., 4000
I_{diff}=the difference between Iₑᵣᵣ and the immediately previous value of Iₑᵣᵣ;
G_{d}=differential gain constant, e.g., −300; and
Vₒᵤₜ=output voltage employed to maintain a predetermined steady target electrical current (e.g., 300 nA).

Algorithm 1 and the aforementioned blocks of hand-held test meter 700 essentially provide a software algorithm-based feedback loop that serves as a constant electrical current driver for a test strip inserted into the hand-held test meter. This software algorithm-based feedback loop employs test strip measured current as an input and generates (along with voltage driver block 708) an applied test strip voltage as the output. The applied test strip voltage is adjusted by software algorithm-based feedback loop to maintain a constant electrical current through the analytical test strip. In doing so, microprocessor block 702 acts in accordance with instructions that are provided as software or firmware that are stored in memory block 704.

The test strip sample detection instructions can employ any suitable averaging algorithm such as the following:

U_{avg}=((N−1)U_{avg}+Uₜ)/N (Algorithm 2)

where:
N=a predetermined averaging constant integer (N can be, for example, equal to 12);
U_{avg}=1.024V (or other suitable predetermined value) when t=1, and thereafter, U_{avg}=U_{avg}′ previously calculated at N−1;
Uₜ=voltage measured across a test strip at time t

The calculation of algorithm 2 can, for example, be performed every 5 milliseconds, with U_{avg}′ feeding back into U_{avg}. When U_{avg}′ is equal to or less than a predetermined threshold value (e.g., 243 mV), the sample detection trigger is activated and an analyte (e.g. glucose) measurement (determination) process started (see steps 840 and 850 of FIG. 8). Algorithm 2 is essentially an averaging algorithm. The minimum amount of time it takes for a sample detection trigger to be met is dependent on the values of the predetermined threshold, U_{avg} for t=1; N, the measured voltage (i.e., Uₜ), and the frequency at which algorithm 2 is performed (for example, every 5 milliseconds which corresponds to a frequency of 200 Hz). However, in a typical but non-limiting use case, once Uₜ is below, and remains below, the predetermined threshold, the sample detection trigger is detected within approximately around 15 measurements.

In a circumstance where Uₜ is physically limited to, for example, 350 mV, and this is not much above an analytical test strip reaction threshold of, for example, 243 mV, a compensation can be provided by having algorithm 1 provide a max Vₒᵤₜ (for example, of 1.024V) that is subsequently limited (by either hardware or software) to, for example, the aforementioned 350 mV. Otherwise, in a simple implementation of hand-held test meters according to the present invention, Uₜ is equal to Vₒᵤₜ and Algorithm 2 is run every time Algorithm 1 calculates a new value of Vₒᵤₜ.

A representative, but non-limiting sequence of steps that can occur during the instruction execution is depicted in FIG. 8. The start of the sequence of FIG. 8 (block 810) is achieved, for example, by the activating (i.e., powering-on) of hand-held test meter 700 and inserting an analytical test strip therein. Step 820 of FIG. 8 can, for example, employ algorithm 1 above while step 830 employs algorithm 2 above. In the flow embodied in FIG. 8, algorithm 1 outputs a voltage (based on an input current) that both drives a current through the test strip and is the voltage input to algorithm 2. The instructions employed in the sequence of steps of FIG. 8 can be wholly or partially embodied in a hand-held test meter as software including, for example, software (also known as a computer program) developed using any suitable programming language known to one skilled in the art including, for example, an object oriented language, C language, C++ language, or a micro-controller code such as assembly language. Moreover, the required software can, for example, be stored in an independent memory block, or in a memory block integrated within a microprocessor block.

FIG. 9 depicts the acceptable match between applied voltages generated by algorithm 1 (marked Drive V S/W) as compared to a more expensive and complex hardware based constant current circuit block (marked Drive V H/W).

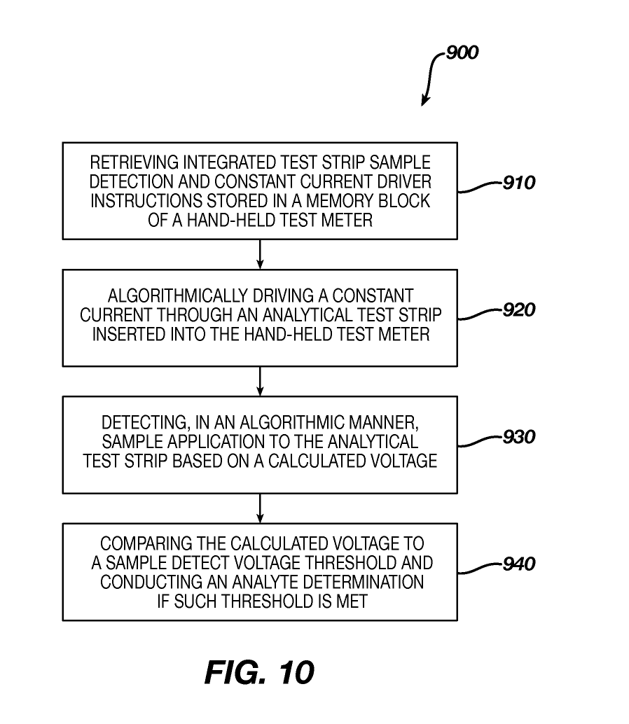

FIG. 10 is a flow diagram depicting stages in a method 900 for operating a hand-held test meter for the determination of an analyte (e.g., glucose) in a bodily fluid sample (for example, a whole blood sample) according to an embodiment of the present invention. Method 900 includes, at step 910, retrieving, using a memory block and a microprocessor block of the hand-held test meter, integrated test strip sample detection and constant current driver instructions stored in the memory block.

At step 920, a constant current is algorithmically driven through an analytical test strip inserted into a strip port connector (SPC) of the hand-held test strip by setting an applied voltage on the strip port connector (SPC) of the hand-held test meter via execution of the integrated test strip detection and constant current driver instructions. Method 900 also includes detecting, in an algorithmic manner, sample application to an analytical test strip inserted in the SPC of the hand-held test meter based on a calculated voltage (see step 930 of FIG. 10).

At step 940, the calculated voltage is compared to a sample detect voltage threshold and if less than such threshold an analyte determination test is conducted. If the sample detect voltage is greater than the threshold, method 900 loops back to step 920 for potential readjustment of the applied voltage and, hence, the current being driven through the analytical test strip.

Once apprised of the present disclosure, one skilled in the art will recognize that methods according to embodiments of the present invention, including method 900, can be readily modified to incorporate any of the techniques, benefits and characteristics of hand-held test meters according to embodiments of the present invention and described herein.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A hand-held test meter for the determination of an analyte in a bodily fluid sample using an analytical test strip, the hand-held test meter comprising:
a microprocessor block;
a strip port connector (SPC);
a voltage driver block operatively connected to the microprocessor block and the SPC;
a current measurement block operatively connected to the SPC and the micro-processor block; and
a memory block operatively coupled to the microprocessor block and storing integrated test strip sample detection and constant current driver instructions,
wherein the memory block, microprocessor block, voltage driver block and current measurement block are configured such that the integrated test strip sample detection and constant current driver instructions, when executed by the microprocessor block, algorithmically detects sample application to a test strip inserted in the SPC and algorithmically drives a constant current through the inserted strip by varying a voltage applied to the SPC by the voltage driver block based on a signal from the current measurement block.

2. The hand-held test meter of claim 1 wherein the constant current driver instructions are loopback control-based instructions.

3. The hand-held test meter of claim 2 wherein the constant current driver instructions are PID loopback control-based instructions.

4. The hand-held test meter of claim 3 wherein the constant current driver instructions are PID loopback control-based instructions of the form:
Vₒᵤₜ=(Iₑᵣᵣ*Gₚ)+(Iᵢₙₜ*Gᵢ)+(I_{diff}*G_{d})
where:
Iₑᵣᵣ=difference between a measured current and a predetermined target current;
Gₚ=a proportional gain constant
Iᵢₙₜ=the sum of previous Iₑᵣᵣ values, however if the measured current is greater than a predetermined over-limit current, then Iᵢₙₜ=0;
Gᵢ=an integral gain constant,
I_{diff}=the difference between Iₑᵣᵣ and the immediately previous value of Iₑᵣᵣ;
G_{d}=a differential gain constant; and
Vₒᵤₜ=output voltage employed to maintain a predetermined steady electrical current.

5. The hand-held test meter of claim 1 wherein test strip sample detection triggers analyte determination.

6. The hand-held test meter of claim 1 wherein the test strip sample detection instructions include an averaging algorithm.

7. The hand-held test meter of claim 6 wherein the averaging algorithm is of the form:
U_{avg}′=((N−1)U_{avg}+Uₜ)/N
where:
N=a predetermined integer;
Uₜ=voltage measured across a test strip at time t;
U_{avg}=the average voltage value of the previous N voltage readings,
U_{avg}′=the newly calculated average voltage incorporating Uₜ

8. The hand-held test meter of claim 1 wherein the analyte is glucose and the bodily fluid sample is a whole blood sample.

9. The hand-held test meter of claim 1 wherein the memory block, microprocessor block, voltage driver block and current measurement block are configured such that the integrated test strip sample detection and constant current driver instructions algorithmically drive a constant current through the inserted strip by outputting a voltage applied to the SPC by the voltage driver block based on a signal from the current measurement block, and
wherein the memory block, microprocessor block, voltage driver block and current measurement block are configured such that the integrated test strip sample detection and constant current driver instructions, when executed by the microprocessor block, algorithmically detects sample application to a test strip inserted in the SPC based on the output voltage applied to the SPC.

10. The hand-held test meter of claim 9 wherein the constant current driver instructions are loopback control-based instructions, and
wherein the test strip sample detection instruction include an averaging algorithm.

11. A method for operating a hand-held test meter for the determination of an analyte in a bodily fluid sample using an analytical test strip, the method comprises:
retrieving, using a memory block and a microprocessor block of the hand-held test meter, integrated test strip detection and constant current driver instructions stored in the memory block;
algorithmically driving a constant current through an analytical test strip inserted into a strip port connector (SPC) of the hand-held test strip by setting an applied voltage on the strip port connector (SPC) of the hand-held test meter by executing the integrated test strip detection and constant current driver instructions; and
detecting, in an algorithmic manner, sample application to a test strip inserted in the SPC based on the applied voltage.

12. The method of claim 11 wherein the detecting of sample application includes:
calculating a sample detect voltage per algorithmic instructions of the integrated test strip detection and constant current driver instructions; and
comparing the calculated sample detect voltage to a predetermined threshold.

13. The method of claim 11 wherein the constant current driver instructions are loopback control-based instructions.

14. The method of claim 13 wherein the constant current driver instructions are PID loopback control-based instructions.

15. The method of claim 14 wherein the constant current driver instructions are PID loopback control-based instructions of the form:
Vₒᵤₜ=(Iₑᵣᵣ*+Gₚ)+(Iᵢₙₜ*Gᵢ)+(I_{diff}*G_{d})
where:
Iₑᵣᵣ=difference between a measured current and a predetermined target current;
Gₚ=proportional gain constant
Iᵢₙₜ=the sum of previous Iₑᵣᵣ values, however if the measured current is greater than a predetermined over-limit current, then Iᵢₙₜ=0;
Gᵢ=integral gain constant
I_{diff}=the difference between Iₑᵣᵣ and the immediately previous value of Iₑᵣᵣ;
G_{d}=differential gain constant; and
Vₒᵤₜ=output voltage employed to maintain a steady electrical current.

16. The method of claim 12 wherein test strip sample detection triggers analyte determination.

17. The method of claim 12 wherein the test strip sample detection instruction include an averaging algorithm.

18. The method of claim 17 wherein the averaging algorithm is of the form:
U_{avg}′=((N−1)U_{avg}+Uₜ)/N
where:
N=a predetermined integer;
Uₜ=voltage measured across a test strip at time t;
U_{avg}=the average voltage value of the previous N voltage readings,
U_{avg}′=the newly calculated average voltage incorporating Uₜ

19. The method of claim 9 wherein the analyte is glucose and the bodily fluid sample is a whole blood sample.

20. The method of claim 11 wherein the constant current driver instructions are loopback control-based instructions, and
wherein the test strip sample detection instructions include an averaging algorithm.
